(19) United States
(12) Patent Application Publication
(10) Pub. No.: US 2005/0118647 A1
(43) Pub. Date: Jun. 2, 2005

(54) Microfluidic mixer apparatus and microfluidic reactor apparatus for microfluidic processing

(76) Inventors: Yung-Chiang Chung, Hsinchu (TW); Ming-Shiung Jan, Hsinchu (TW); Ju-Hwa Lin, Hsinchu (TW); Shr-Hau Huang, Hsinchu (TW)

Correspondence Address: SHAW PITTMAN;IP GROUP, 1650 TYSONS BOULEVARD, SUITE 1300, MCLEAN, VA, 22102, US

(21) Appl. No.: 10/986,818

(22) Filed: Nov. 15, 2004

Related U.S. Application Data
(63) Continuation-in-part of application No. 10/038,994, filed on Dec. 31, 2001.

(51) Int. Cl. G01N 33/53

(52) U.S. Cl. 435/7.1

(57) ABSTRACT

A device for extracting nucleic acids includes a cover including a first opening and a second opening spaced apart from the first opening, a chip including a first terminal to communicate with the first opening and a second terminal to communicate with the second opening, and a flow channel of the chip including an inner surface onto which immobilized beads are dispersed for binding with nucleic acids in a fluid flowing in the flow channel.

This application is a continuation-in-part of U.S. patent application Ser. No. 10/038,994, filed Dec. 31, 2001, which is herein incorporated by reference in its entirety.

### BACKGROUND

1. Field of the Invention

The present invention relates generally to microfluidic apparatus employed for microfluidic processing. More particularly, the present invention relates to microfluidic mixer apparatus and microfluidic reactor apparatus employed for microfluidic processing.

2. Background of the Invention

Microelectronic fabrications are formed from microelectronic substrates within and upon which are formed microelectronic devices and over which are formed patterned microelectronic conductor layers which are separated by microelectronic dielectric layers.

As an extension of microelectronic fabrication technology, there has recently evolved a branch of technology generally referred to as microelectromechanical system (MEMS) technology. MEMS technology generally employs microfabrication techniques which are analogous with those employed within microelectronic fabrication technology, but where a resulting MEMS product (in comparison with a microelectronic fabrication product) possess microelectromechanical capabilities rather purely microelectronic capabilities or optoelectronic microelectronic capabilities. Within the general field of MEMS technology considerable interest has arisen in microfluidic MEMS products which require the fabrication of micro-pumps, micro-valves and micro-channels within a substrate, such as to effect various microfluidic operations within the microfluidic MEMS products.

Applications of microfluidic MEMS products are often directed towards testing, evaluating or screening of large numbers of sample material fluids while employing limited volumes of the sample material fluids.

While microfluidic MEMS products thus provide a basis for several desirable analytical tools which may effect further advances in other technology fields, microfluidic MEMS products are nonetheless not entirely without problems.

In that regard, microfluidic MEMS products, and their components, are often difficult to readily fabricate and operate.

It is thus desirable in the art of microfluidic MEMS technology to provide microfluidic MEMS components which readily fabricated and operated.

It is towards the foregoing object that the present invention is directed.

Various microfluidic MEMS products having desirable properties, and components thereof, have been disclosed in the art of microfluidic MEMS technology.

Included among the microfluidic MEMS products and components thereof, but not limited among the microfluidic MEMS products and components thereof, are microfluidic MEMS products and components thereof disclosed within: (1) Desai et al., in U.S. Pat. No. 5,921,678 (a microfluidic MEMS mixer component capable of initiating or quenching chemical reactions with intervals as short as 100 microseconds, where the microfluidic MEMS mixer component comprises a plurality of “T” shaped channels where separate pairs of reagents meet head-on to mix at an apex of a “T” and exit through a base of the “T”); (2) Furcht et al., in U.S. Pat. No. 6,054,277 (a fully integrated microfluidic MEMS product employed for testing genetic material, where the fully integrated microfluidic MEMS product provides for separation of genetic material as well as amplification of genetic material); (3) Lee et al., in U.S. Pat. No. 6,146,103 (a magnetohydrodynamic micro-pump and micro-sensor component which may be employed within a microfluidic MEMS product, wherein the magnetohydrodynamic micro-pump and micro-sensor component may be fabricated provide reversible microfluid flow, as well as microfluid mixing); and (4) Henderson et al., in U.S. Pat. No. 6,258,263 (a microfluidic MEMS product fabricated such as to provide a liquid chromatograph microfluidic MEMS product).

The teachings of each of the foregoing references is incorporated herein fully by reference.

Desirable in the art of microfluidic MEMS technology are additional microfluidic MEMS components which may be readily fabricated and operated.

It is towards the foregoing object that the present invention is directed.

### BRIEF SUMMARY OF THE INVENTION

The present invention is directed to a circuit and a method that obviate one or more problems resulting from the limitations and disadvantages of the prior art.

In accordance with an embodiment of the present invention, there is provided a device for extracting nucleic acids that comprises a chip, and a flow channel of the chip including immobilized beads dispersed therein for extracting the nucleic acids.

Also in accordance with the present invention, there is provided a device for extracting nucleic acids that comprises a cover including a first opening and a second opening spaced apart from the first opening, and a chip including a first terminal to communicate with the first opening and a second terminal to communicate with the second opening, the chip including a flow channel disposed between the first terminal and the second terminal, wherein the flow channel includes an inner surface onto which immobilized beads are dispersed for binding with nucleic acids in a fluid flowing in the flow channel.

Further in accordance with the present invention, there is provided a device for extracting nucleic acids that comprises a chip including a flow channel, a cover disposed over the chip including a first opening and a second opening diagonally disposed with respect to the first opening, and a mixer disposed between the chip and the cover for mixing a sample and a buffer, wherein the flow channel includes immobilized beads dispersed therein for extracting nucleic acids in a fluid containing the sample and the buffer.

Still in accordance with the present invention, there is provided a method for extracting nucleic acids that comprises providing a chip, providing the chip with a flow channel including an inner surface, and dispersing immobilized beads for extracting the nucleic acids on the inner surface of the flow channel.

Yet still in accordance with the present invention, there is provided a method for extracting nucleic acids that comprises providing a flow channel, providing a solution of bead powder and deionized water, injecting the solution of the bead powder and deionized water into the flow channel, and shaking the solution in the flow channel to disperse immobilized beads in the flow channel.

Additional features and advantages of the present invention will be set forth in part in the description which follows, and in part will be obvious from the description, or may be learned by practice of the invention. The features and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

The accompanying drawings, which are incorporated in and constitute a part of this specification, illustrate one embodiment of the present invention and together with the description, serves to explain the principles of the invention.

### BRIEF DESCRIPTION OF THE DRAWINGS

Reference will now be made in detail to the present embodiment of the invention, an example of which is illustrated in the accompanying drawings. Wherever possible, the same reference numbers are used throughout the drawings to refer to the same or like parts.

FIG. 1 is a schematic top view of a microfluidic mixing apparatus in accordance with one embodiment of the present invention;

FIG. 2 is a schematic cross-sectional view of the microfluidic mixing apparatus shown in FIG. 1;

FIG. 3 is a schematic top view of a microfluidic reactor in accordance with one embodiment of the present invention;

FIG. 4 is a cross-sectional view of the microfluidic reactor shown in FIG. 3;

FIG. 5 is another cross-sectional view of the microfluidic reactor shown in FIG. 3;

FIG. 6A is a cross-sectional view of a device for extracting nucleic acids in accordance with one embodiment of the present invention;

FIG. 6B is a schematic top view of a cover of the device shown in FIG. 6A;

FIG. 6C is a schematic top view of a microfluidic chip of the device shown in FIG. 6A;

FIG. 6D is another cross-sectional view of the device shown in FIG. 6A;

FIG. 7A is a cross-sectional view of a device for extracting nucleic acids in accordance with another embodiment of the present invention;

FIG. 7B is a top view of a cover of the device shown in FIG. 7A;

FIG. 8A is a diagram showing experiment results of extraction; and

FIG. 8B is a diagram showing a comparison of the experiment results shown in FIG. 8A.

### DETAILED DESCRIPTION OF THE INVENTION

The present invention provides a pair of microfluidic microelectromechanical system (MEMS) components, wherein each of the pair of microfluidic MEMS components is readily fabricated and operated.

The present invention realizes the foregoing objects with respect to a microfluidic mixer apparatus component for use within a MEMS product by fabricating the microfluidic mixer apparatus component, which comprises: (1) a substrate having formed therein an aperture; and (2) at least two channels also formed within the substrate such as to terminate at the aperture, such that the at least two channels which terminate at the aperture terminate obliquely with respect to the aperture such as to effect a swirling mixing of at least two reagents introduced into the aperture through the at least two channels.

The present invention realizes the foregoing object with respect to a microfluidic reactor apparatus component for use within a MEMS product, where: (1) the microfluidic reactor apparatus also comprises a substrate having formed therein an aperture, further where; (2) the aperture has a first end portion contiguous with a middle portion in turn contiguous with a second end portion, by fabricating within the middle portion of the aperture, but not the first end portion of the aperture or the second end portion of the aperture, at least one baffle which intrudes into the aperture.

While the preferred embodiments of the present invention provide: (1) a microfluidic mixing apparatus (and a method for operation of the microfluidic mixing apparatus); and (2) a microfluidic reactor apparatus (and a method for operation of the microfluidic reactor apparatus), wherein each of the foregoing pair of microfluidic apparatus provides particular value within the context of testing, evaluation and screening of pharmaceutical materials and genetic materials (including nucleic acids and proteins) the pair of microfluidic apparatus of the present invention (and their methods for operation thereof) may be employed within microfluidic MEMS applications other than pharmaceutical, genetic, biochemical and biomedical applications. Such other applications may include, but are not limited to, agrichemicals formulation applications and materials engineering applications.

Microfluidic Mixing Apparatus

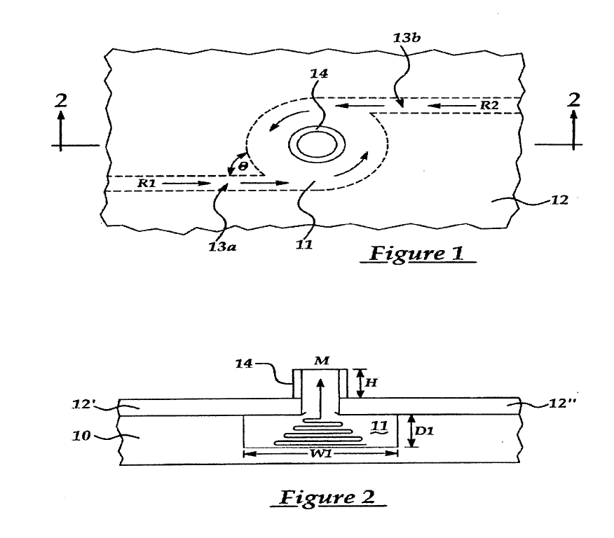

Referring now to FIG. 1 and FIG. 2 there is shown a schematic plan-view diagram and a schematic cross-sectional diagram illustrating a microfluidic mixing apparatus in accord with a first preferred embodiment of the present invention. The microfluidic mixing apparatus may be employed within a microfluidic MEMS product.

Shown in FIG. 1 is a schematic plan-view diagram of the microfluidic mixing apparatus.

Shown in FIG. 1 is a cover plate 12 which covers a substrate (which is not specifically illustrated) which in part comprises the microfluidic mixing apparatus of the present invention. As is further illustrated within the schematic plan-view diagram of FIG. 1, and as is formed within the substrate beneath the cover plate 12, is an aperture 11 into which in turn terminates at least a pair of reagent supply channels 13a and 13b which is also formed within the substrate. As is further illustrated within the schematic plan-view diagram of FIG. 1, each of the pair of reagent supply channels 13a and 13b terminates at the aperture 11 obliquely such that when a pair of reagents R1 and R2 is introduced into the aperture 11 through the corresponding pair of reagent supply channels 13a and 13b, the pair of reagents R1 and R2 mixes in a swirling fashion. As is finally illustrated within the schematic plan-view diagram of FIG. 1, there is shown an outlet port 14 which allows a mixture of the pair of reagents R1 and R2 to exit from the aperture 11 in a direction perpendicular to a plane of the substrate.

Shown in FIG. 2 is a schematic cross-sectional diagram of a microfluidic mixer apparatus corresponding with the microfluidic mixer apparatus whose schematic plan-view diagram is illustrated in FIG. 1.

Shown in FIG. 2 is the substrate 10 having formed therein the aperture 11. The substrate 10, including the aperture 11, in turn is covered by and has assembled thereto the cover plate 12′/12″, which further in turn has assembled thereto the outlet port 14 from which may exit a mixture M (such as a homogeneous solution) of the reagents R1 and R2 after having been mixed within the aperture 11.

Within the first preferred embodiment of the present invention with respect to the substrate 10, the substrate 10 may be fabricated from a material selected from the group including but not limited to conductor materials, semiconductor materials and dielectric materials, as well as laminates thereof. Within the first preferred embodiment of the present invention, however, the substrate 10 is typically and preferably an inorganic substrate, such as but not limited to a semiconductor substrate, typically and preferably formed to a thickness of from about 4 to about 6 mm. Similarly, within the present invention the cover plate 12 and the outlet port 14 may also be formed from materials selected from the group including but not limited to conductor materials, semiconductor materials and dielectric materials, although within the first preferred embodiment of the present invention both the cover plate 12 and the outlet port 14 are preferably formed of transparent materials, such as but not limited to transparent glass materials, so that operation of the microfluidic mixing apparatus of the first preferred embodiment of the present invention may be visually inspected. Typically and preferably, the cover plate 12 is formed to a thickness of from about 4 to about 6 mm and the outlet port 14 provides a protrusion height H (as illustrated within FIG. 2) upon cover plate 12 of from about 4 to about 6 mm.

Within the preferred embodiment of the present invention with respect to the aperture 11, the aperture 11 typically and preferably has an aperture width W1 (as illustrated within FIG. 2) within the substrate 10 of from about 3 to about 5 mm and an aperture depth D1 (as illustrated within FIG. 2) within the substrate 10 of from about 0.4 to about 0.6 mm. Similarly, although the preferred embodiment of the present invention illustrates the aperture 11 as a circular shaped aperture, within the present invention the aperture 11 may be provided in shapes selected from the group including but not limited to circular shapes, elliptical shapes, irregular continuous sided shapes and polygonal shapes (i.e., discontinuous sided shapes), presuming that at least a pair of reagent supply channels is properly terminally disposed with respect to the aperture such as to effect a swirling mixing of at least a pair of reagents introduced into the aperture, in accordance with the present invention.

As is illustrated within the schematic plan-view diagram of FIG. 1, in order to provide the foregoing swirling mixing of the pair of reagents R1 and R2, each of the pair of reagent supply channels 13a and 13b terminates at the aperture 11 with a tangential, oblique and acute angle of incidence θ of from about 20 to about 80 degrees. Additionally, each of the pair of reagent supply channels 13a and 13b is formed of a line width within the substrate 10 of from about 0.4 to about 0.6 mm and a depth within the substrate 10 of from about 0.4 to about 0.6 mm. For ease in manufacturing and for more optimal performance, the depth D1 of the aperture 11 within the substrate 10 is typically equivalent with the depth of each of the pair of reagent supply channels 13a and 13b within the substrate 10, although such is not required within the present invention. Finally, although the preferred embodiment of the present invention illustrates the present invention within the context of two reagent supply channels 13a and 13b terminating at the aperture 11, the present invention also contemplates that there may be a greater number of reagent supply channels terminating at the aperture 11. Such an increased number of reagent supply channels will typically and preferably be equally spaced with respect to the periphery of the aperture 11.

Within the first preferred embodiment of the present invention, the aperture 11, as well as the pair of reagent supply channels 13a and 13b, may be formed while employing etching and laminating fabrication methods as are otherwise generally conventional in the art of microfluidic MEMS fabrication.

Within the preferred embodiment of the present invention with respect to the outlet port 14, the outlet port 14 is typically and preferably substantially centered (within the limits of fabrication and assembly technology, i.e., within about ±10% centering uniformity) with respect to the aperture 11, and provided with an outlet port 14 opening line width of from about 1 to about 2 mm.

With respect to operation of the microfluidic mixer apparatus of the first preferred embodiment of the present invention as illustrated within the schematic plan-view diagram of FIG. 1 and schematic cross-sectional diagram of FIG. 2, and within the context of the foregoing dimensions for the aperture 11, the pair of reagent supply channels 13a and 13b and the outlet port 14, there is typically and preferably provided a reagent R1 and reagent R2 flow of from about 5 to about 30 microliters per second into the aperture 11, although a reagent R1 flow need not be equivalent with a reagent R2 flow.

Upon fabricating and operating a microfluidic mixer apparatus in accordance with the schematic plan-view diagram of FIG. 1 and the schematic cross-sectional diagram of FIG. 2, there is fabricated and operated a microfluidic mixer apparatus in accord with the first preferred embodiment of the present invention. The microfluidic mixer apparatus provides for efficient fabrication and operation insofar as the microfluidic mixer apparatus provides for a swirling mixing of at least two reagents supplied into an aperture which comprises in part the microfluidic mixer apparatus.

Microfluidic Reactor Apparatus

Referring now to FIG. 3 to FIG. 5, there is shown a schematic plan-view diagram and a pair of schematic cross-sectional diagrams illustrating a microfluidic reactor apparatus in accordance with a second preferred embodiment of the present invention. The microfluidic reactor apparatus may also be employed within a microfluidic MEMS product.

Shown in FIG. 3 is a schematic plan-view diagram of the microfluidic reactor apparatus.

Analogously with the microfluidic mixer apparatus whose schematic plan-view diagram is illustrated in FIG. 1, there is shown within the microfluidic reactor apparatus whose schematic plan-view diagram is illustrated in FIG. 3 a cover plate 22 assembled to and covering a substrate (which is also not specifically illustrated), wherein the cover plate 22 in turn has assembled thereto a pair of inlet/outlet ports 24a and 24b. Also similarly with the microfluidic mixer apparatus in accord with the schematic plan-view diagram of FIG. 1 and the schematic cross-sectional diagram of FIG. 2, the microfluidic reactor apparatus as illustrated within the schematic plan-view diagram of FIG. 3 also comprises an aperture 21 formed within the substrate. Within the second preferred embodiment of the present invention, and as illustrated within the schematic plan-view diagram of FIG. 3, the aperture 21 comprises three portions:

(1) a first end portion P1, the first end portion P1 being contiguous with; (2) a middle portion P2, the middle portion P2 in turn being contiguous with; (3) a second end portion P3.

As is finally illustrated within the schematic cross-sectional diagram of FIG. 3, the middle portion P2 of the aperture 21, but neither the first end portion P1 of the aperture 21 nor the second end portion P3 of the aperture 21, has formed intruding therein a pair of baffles 23a and 23b. Within the second preferred embodiment of the present invention, the pair of baffles 23a and 23b serves to facilitate operation of the microfluidic reactor apparatus of the second preferred embodiment of the present invention.

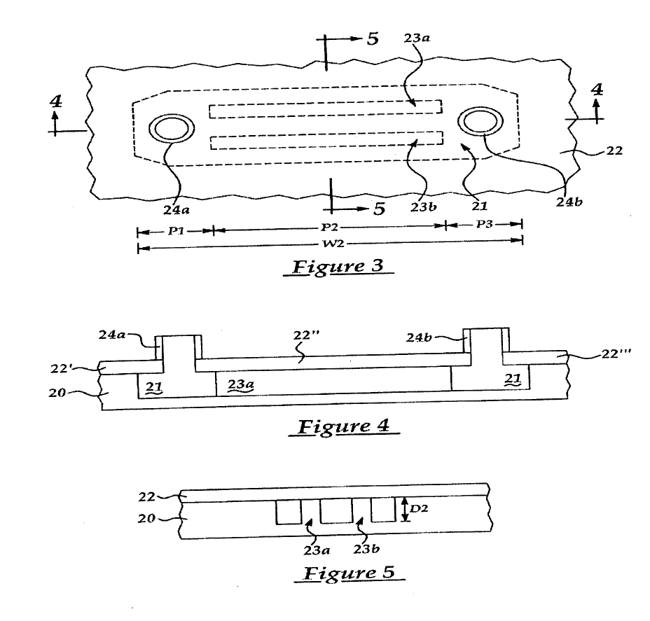

Shown in FIG. 4 is a first schematic cross-sectional diagram of a microfluidic reactor apparatus corresponding with the microfluidic reactor apparatus whose schematic plan-view diagram is illustrated in FIG. 3.

Shown in FIG. 4 is the substrate 20 having assembled thereto the cover plate 22′/22″/22′″ in turn having assembled thereto the pair of inlet/outlet ports 24a and 24b. Similarly, the schematic cross-sectional diagram of FIG. 4 also illustrates the aperture 21 having intruding therein the baffle 23a.

Shown in FIG. 5 is a second schematic cross-sectional diagram of a microfluidic reactor apparatus corresponding with the microfluidic reactor apparatus whose schematic plan-view diagram is illustrated in FIG. 3.

Also shown within the schematic cross-sectional diagram of FIG. 5 is the substrate 20 having assembled thereto the cover plate 22. Similarly, and also illustrated within the schematic cross-sectional diagram of FIG. 5 is the pair of baffles 23a and 23b which may derive from incomplete etching of the substrate 20, when forming the aperture 21. Alternatively, or an adjunct, the pair of baffles 23a and 23b may be formed as part of the cover plate 22, or as an independent component which is assembled within the aperture 21 when forming the microfluidic reactor apparatus in accordance with the second preferred embodiment of the present invention. Although the pair of baffles 23a and 23b is illustrated as completely spanning from the substrate 20 to the cover plate 22, such is not required within the present invention. Thus, within a microfluidic reactor apparatus in accordance with the present invention a series of baffles may provide a series of gaps which separate the series of baffles from the substrate 20, the cover plate 22 or both the substrate 20 and the cover plate 22.

Within the second preferred embodiment of the present invention with respect to the substrate 20, the cover plate 22 and the pair of inlet/outlet ports 24a and 24b, the substrate 20, the cover plate 22 and the pair of inlet/outlet ports 24a and 24b may be formed employing materials and dimensions analogous or equivalent to the materials and dimensions employed for forming the substrate 10, the cover plate 12 and the outlet port 14 within the microfluidic mixer apparatus in accord with the first preferred embodiment of the present invention.

Within the second preferred embodiment of the present invention with respect to the aperture 21, the aperture 21 typically and preferably has a longitudinal line width W2 (as illustrated in FIG. 3) of from about 500 to about 1000 mm, where the longitudinal line width includes the first end portion P1, the middle portion P2 and the second end portion P3 of the aperture 21. Similarly, the first end portion P1 and the second end portion P3 of the aperture 21 each have a longitudinal line width of from about 150 to about 250 mm and the middle portion P2 of the aperture has a longitudinal line width of from about 400 to about 500 mm. Finally, the aperture 21 typically and preferably has a lateral line width within the substrate 20 (which transects the pair of baffles 23a and 23b) of from about 4 to about 6 mm and a depth D2 within the substrate 20 (as illustrated within the schematic cross-sectional diagram of FIG. 5) of from about 0.5 to about 1.5 mm.

Within the preferred embodiment of the present invention with respect to the pair of baffles 23a and 23b, each of the pair of baffles 23a and 23b typically and preferably has a lateral line width of from about 0.5 to about 1.5 mm and is separated by a sub-aperture channel distance of from about 0.5 to about 1.5 mm. Although the second preferred embodiment of the present invention illustrates the microfluidic reactor apparatus as comprising the aperture 21 having formed therein two baffles 23a and 23b, a microfluidic reactor apparatus in accord with the present invention may have additional baffles formed within an aperture which comprises the microfluidic reactor apparatus, but will have at least one baffle.

With respect to operation of the microfluidic reactor apparatus of the second preferred embodiment of the present invention, the aperture 21, including the baffles 23a and 23b, typically and preferably has contained therein, and preferably also immobilized therein, a sorbtive material for sorbtion and desorbtion of a sample material of interest which is desired to be separated while employing the microfluidic reactor apparatus in accordance with the second preferred embodiment of the present invention. Thus, the microfluidic reactor apparatus of the present invention is typically and preferably employed as a microfluidic liquid chromatography apparatus.

Within the second preferred embodiment of the present invention, the sorbtive material is of a composition as is otherwise generally conventional in the art of liquid chromatography, and the sorbtive material may similarly be immobilized within the aperture 21 and upon the baffles 23a and 23b while employing immobilization methods, such as chemical immobilization methods and physical immobilization methods, as are otherwise generally conventional in the art of liquid chromatograph.

When employed for sorbing and separating nucleic acid polymers and protein polymers, sorbants will typically and preferably comprise amino (i.e., —NH2) or cyano (i.e., —CN) functionality. Appropriate commercially available sorbants for such biochemical applications may include, but are not limited to:

(1) MagicBead #1 (Magic Bead Corp.); (2) QIAEX II Suspension #20902 (QIAGEN Corp.); (3) GENECLEAN SPIN GLASSMILK #1101-201 (Q.BIOgene Corp.); and (4) NucleoTrap Suspension #4080-1 (Clontech Corp.)

With respect to operation of the microfluidic reactor apparatus of the present invention, and within the context of aperture 21 and baffle 23a and 23b dimensions disclosed above, there is typically and preferably provided a flow rate of a sample solution through the input/outlet ports 24a and 24b of from about 10 to about 40 microliters per second. Similarly, to facilitate optimal sorbtion of a target material from a sample solution onto a sorbant, the sample solution may be cycled sequentially and reversibly through the microfluidic reactor apparatus of the second preferred embodiment of the present invention.

As is further understood by a person skilled in the art, by fabricating the microfluidic reactor apparatus of the second preferred embodiment of the present invention with the aperture 21 having the first end portion P1 and the second end portion P3 which do not have intruding therein the baffles 23a and 23b, the microfluidic reactor apparatus of the present invention is readily fabricated and operated insofar as the microfluidic reactor apparatus provides minimal difficulty with respect to alignment of the cover plate 22, or the inlet/outlet ports 24a and 24b, with a series of channels defined by the pair of baffles 23a and 23b.

Upon fabricating and operating the microfluidic reactor apparatus of the second preferred embodiment of the present invention, there is provided the microfluidic reactor apparatus which is, for the foregoing reasons, readily fabricated and operated.

In another embodiments, the present invention provides a device and method for extracting nucleic acids. Nucleic acids, including deoxyribonucleic acid (“DNA”) and ribonucleic acid (“RNA”), are the informational molecules of all living organisms. DNA sequencing and analysis have been developed in a chip format by DNA hybridization. The amplification of DNA has become faster because of the improvement in micro polymerase chain reaction (“PCR”) systems. Sample preparation is an important process for DNA sequence analysis and genetic recombination. Automation of sample preparation process is required in experimental and clinical fields. It involves cell lysis, DNA binding and release. The quality of extraction is a basic criterion for successful application in this field. Beads and buffers for DNA extraction have been developed to be commercial products. They provide the advantages of quick processing time, reduced chemical requirements, easy separation from the beads and ease of automation.

Conventional methods for DNA extraction generally include column-based techniques and centrifugation or precipitation techniques. The conventional methods have the disadvantage of being time consuming, difficult to automate or difficult to scale down to small sample volumes. At the microscales, high concentration solutions, for example, whole blood, will impede fluid flow such that the process becomes time-consuming. In particular, the centrifugation and precipitation processes are difficult to perform in chips. Even though the DNA-extracting beads have been developed, the residual solution cannot be totally exhausted because the beads are free to move. This also means that the beads can be easily exhausted at the same time. It is therefore desirable to have a device and method for improving the DNA extraction using immobilized beads and flowing solution back and forth.

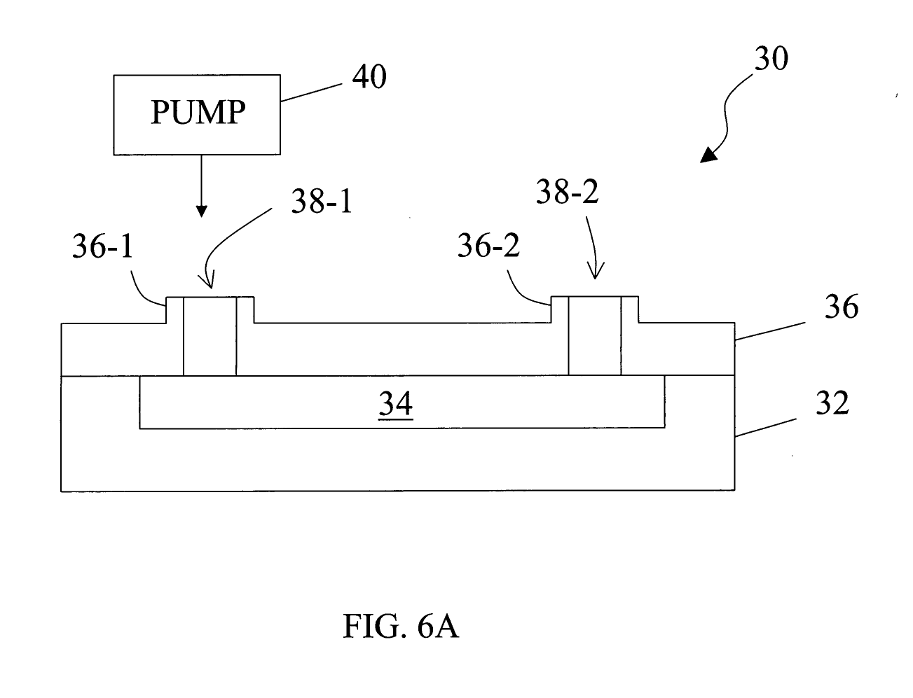

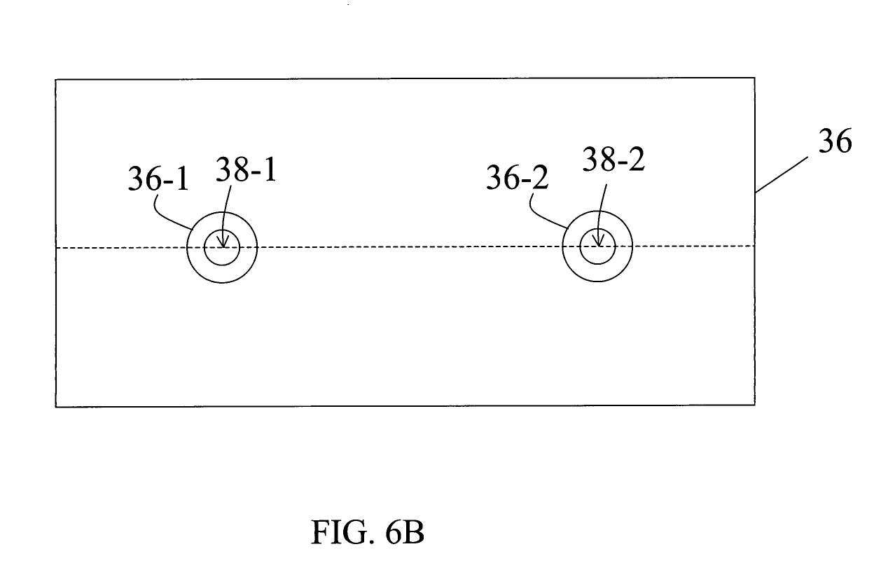

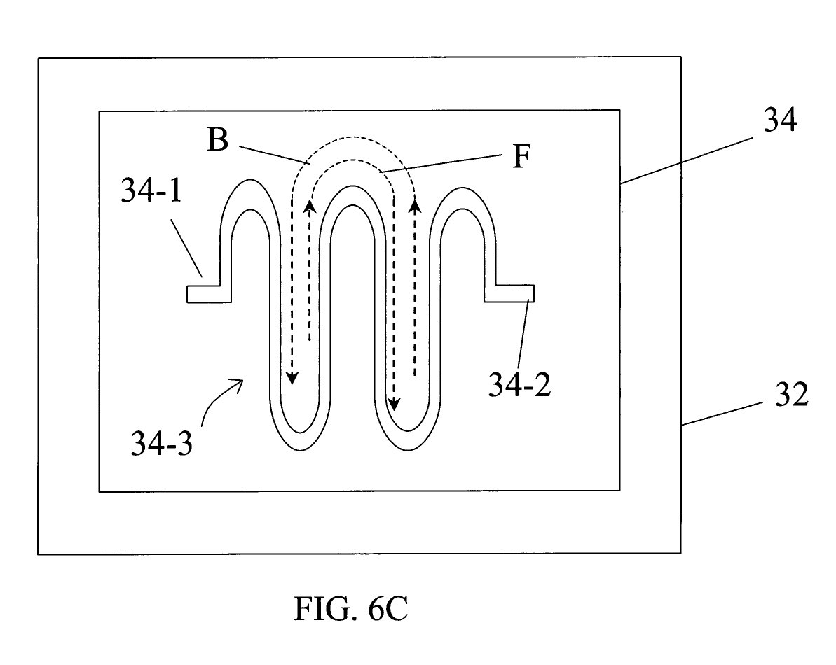

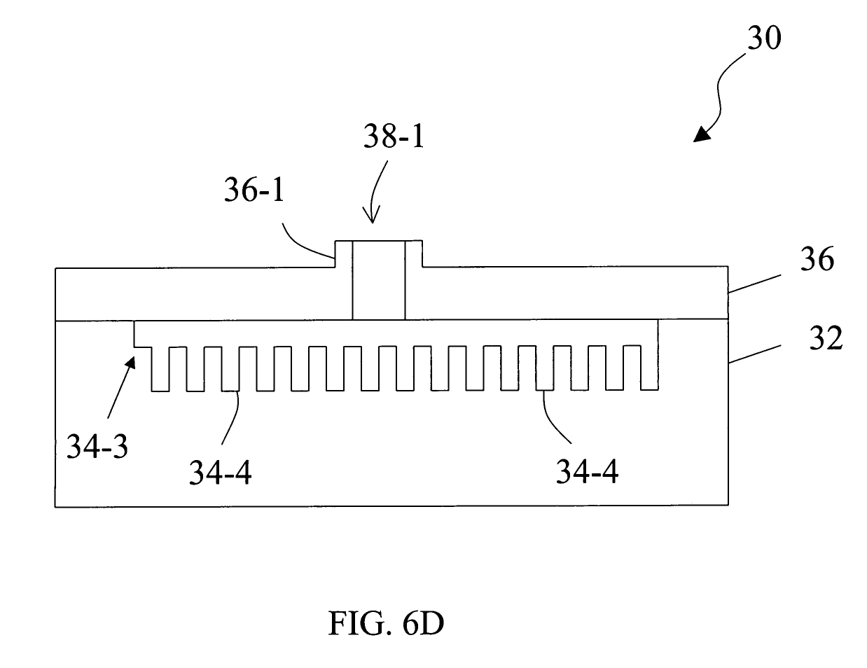

FIG. 6A is a cross-sectional view of a device 30 for extracting nucleic acids in accordance with one embodiment of the present invention. Referring to FIG. 6A, device 30 includes a microfluidic chip 32 and a cover 36. Microfludic chip 32, for example, a poly-methylmethacrylate (“PMMA”) substrate, includes a region 34 where a flow channel is fabricated. Cover 36 includes a first pedestal 36-1 and a second pedestal 36-2 where a first opening 38-1 and a second opening 38-2 are provided, respectively.

FIG. 6B is a schematic top view of cover 36 of device 30 shown in FIG. 6A. First pedestal 36-1 and second pedestal 36-2 facilitate injection of a sample and a buffer into microfluidic chip 34 through first opening 38-1 and second opening 38-2, respectively. The sample is mixed and treated with the buffer in microfluidic chip 34. In other embodiments, the sample and buffer are mixed together into a fluid, which is then injected through first opening 38-1 and second opening 38-2. In one embodiment according to the present invention, also referring to FIG. 6A, a driving apparatus 40 such as a pump or syringe is used to induce oscillations to the fluid flow in microfluidic chip 34. In one aspect, the driving apparatus 40, disposed on one of first pedestal 36-1 or second pedestal 36-2, provides a bi-directional flow of the fluid in, for example, a forward direction and a backward direction, as shown in FIG. 6C, indicated by dashed-line paths F and B, respectively.

FIG. 6C is a schematic top view of microfluidic chip 34 of device 30 shown in FIG. 6A. Referring to FIG. 6C, microfluidic chip 34 includes a flow channel 34-3 therein winding its path from a first terminal 34-1 to a second terminal 34-2 thereof. First terminal 34-1 communicates with first opening 38-1 to receive one of the sample or buffer. Second terminal 34-2 communicates with second opening 38-2 to receive the other of the sample or buffer. The inner surface of flow channel 34-3 is dispersed with immobilized beads or micro-carriers (not shown). The beads used for DNA extraction may be obtained from, for example, Magic Bead Inc. (USA). To immobilize the beads in flow channels 34-3, in accordance with a method of the present invention, one gram of bead powder is added and dispersed into, for example, 20 ml (milliliter) of distilled deionized water. Next, a portion of the solution, for example, 50 μl (micro-litter), is injected into flow channel 34-3. After an hour of mild shaking, most of the beads in the solution are immobilized onto flow channel 34-3. In one embodiment according to the present invention, the ratio of the surface area of immobilized beads to that of free beads is approximately 0.7. Nucleic acids can bind to the kinds of beads whose surfaces have organic groups. When the ion concentration of the solution is changed, the bound nucleic acids can be released into the solution.

FIG. 6D is another cross-sectional view of device 30 shown in FIG. 6A. Referring to FIG. 6D, flow channel 34-3 includes projecting portions 34-4 along its path formed into microfluidic chip 32. Projecting portions 34-4, also dispersed with immobilized beads, help increase the probability of collision between the beads and nucleic acids. Moreover, the driving apparatus for flowing the fluid backward and forward in flow channel 34-3 also helps increase the probability of collision between the immobilized beads and the nucleic acids.

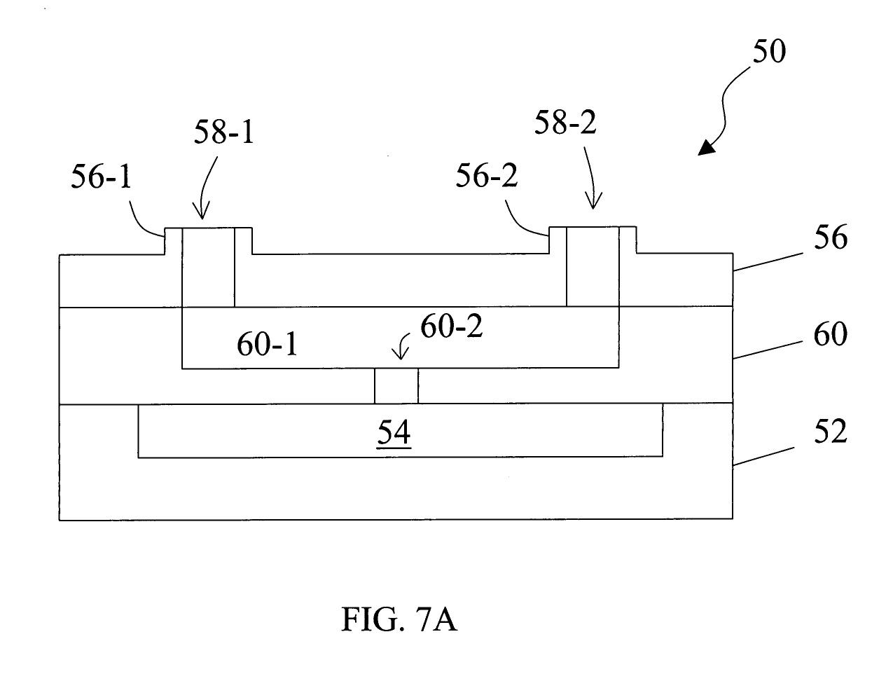

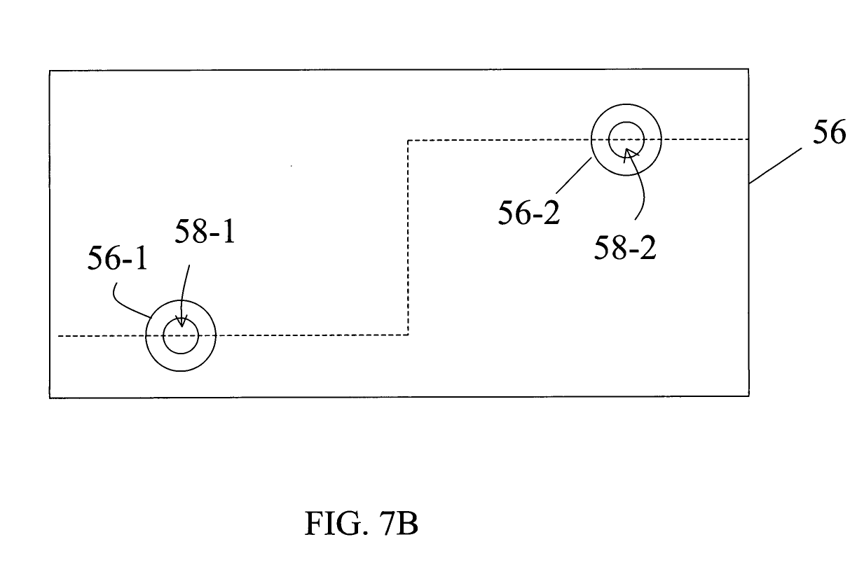

FIG. 7A is a cross-sectional view of a device 50 for extracting nucleic acids in accordance with another embodiment of the present invention. Referring to FIG. 7A, device 50 includes a microfluidic chip 52, a mixing layer 60, and a cover 56. Microfluidic chip 52, for example, a PMMA substrate, includes a region 54 where a flow channel is fabricated. Mixing layer 60 includes a mixer 60-1 for providing a fluid by mixing a sample and a buffer. Cover 56 includes a first pedestal 56-1 into which a first opening 58-1 is provided to communicate a first terminal (not numbered) of mixer 60-1, and a second pedestal 56-2 into which a second opening 58-2 is provided to communicate with a second terminal (not numbered) of mixer 60-1. Mixer 60-1 has an outlet 60-2 provided in a bottom surface thereof to communicate with region 54 of microfluidic chip 52. Mixer 60-1 together with outlet 60-2 is similar in structure to the microfluidic mixing apparatus together with outlet port 14 shown in FIG. 1. In one embodiment according to the present invention, mixing layer 60 and cover 56 are removable from device 50 so that fluid can be prepared by mixing a sample in a buffer in a remote mixing layer 60 before the fluid is injected into microfluidic chip 52.

FIG. 7B is a top view of cover 56 of device 50 shown in FIG. 7A. Referring to FIG. 7B, first pedestal 56-1 and second pedestal 56-2, and in turn first opening 58-1 and second opening 58-2, are diagonally disposed, or offset, with respect to one another on cover 56. As a result, as shown in FIG. 1, a swirl is generated when a sample and a buffer are injected from first opening 58-1 and second opening 58-2, or vice versa, respectively.

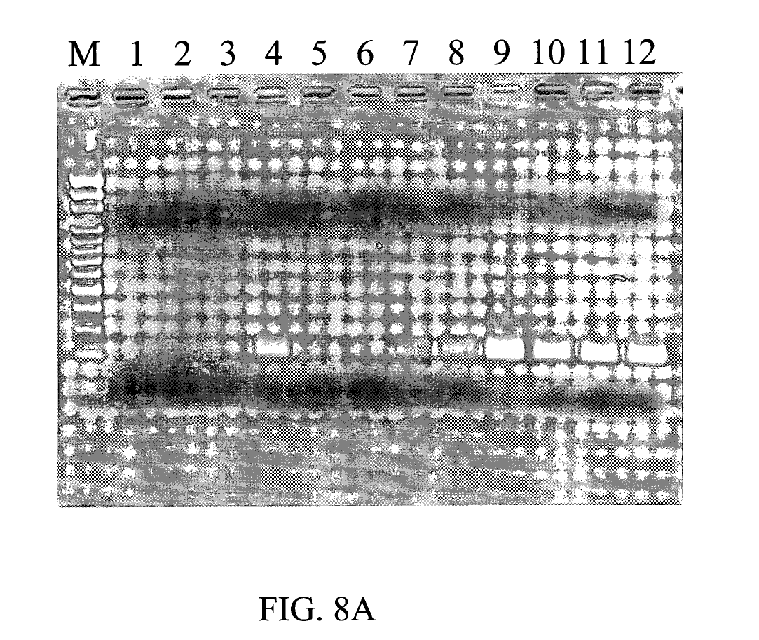

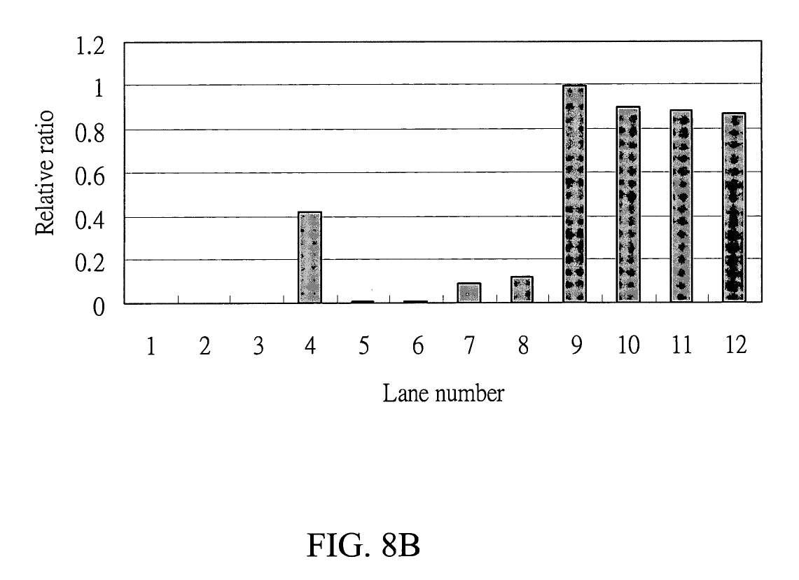

FIG. 8A is a diagram showing experiment results of extraction. Referring to FIG. 8A, gel electrophoresis fluorescence images of the DNA extracted from 10⁵ Escherichia coli (hereinafter the “E. coli) cells of 75 μl of solution after PCR amplification are used to compare the extraction efficiencies using both immobilized and free beads. Since the molecular weight of genomic DNA in E. coli cells is about 3.1×10⁹ daltons (g mol⁻¹), there is about 0.5 ng (nano gram) genomic DNA in 10⁵ E. coli cells. The experimental conditions are summarized in Table 1.

TABLE 1

| Lane Number | 1 | 2 | 3 | 4 | 5 | 6 | 7 | 8 | 9 | 10 | 11 | 12 |
|---|---|---|---|---|---|---|---|---|---|---|---|---|
| Bead | No¹ | No | No | Fr² | Fr | Fr | Fr | Fr | No | Im³ | Im | Im |
| Condition | | | | | | | | | | | | |
| Container | Dvc⁴ | Dvc | Dvc | Dvc | Dvc | Dvc | Dvc | Dvc | Epd⁵ | Dvc | Dvc | Dvc |
| Fluid | Fl⁶ | Fl | Fl | Fl | Fl | Fl | Fl | St⁷ | Vrt⁸ | Fl | Fl | Fl |
| Condition | | | | | | | | | | | | |
| E. coli | 10⁵ | 10⁵ | 10⁵ | 10⁵ | 10⁵ | 10⁵ | 5 × 10⁴ | 10⁵ | 0 | 10⁵ | 10⁵ | 10⁵ |
| Cell Number | | | | | | | | | | | | |
| Weight of E. coli | 0 | 0 | 0 | 0 | 0 | 0 | 0 | 0 | 0.5 | 0 | 0 | 0 |
| Genomic | | | | | | | | | | | | |
| DNA (ng) | | | | | | | | | | | | |
| Serum | 0 | 25 | 50 | 0 | 25 | 50 | 0 | 0 | 0 | 0 | 25 | 50 |
| Volume (μl) | | | | | | | | | | | | |
| Total Volume | 75 | 75 | 75 | 75 | 75 | 75 | 75 | 75 | 75 | 75 | 75 | 75 |
| (μl) | | | | | | | | | | | | |

¹No: no beads;
²Fr: free beads;
³Im: immobilized beads;
⁴Dvc: device;
⁵Epd: Eppendorf;
⁶Fl: flow back and forth;
⁷St: static;
⁸Vrt: vortex;

E. coli is cultured in 5 ml of LB medium (NaCl: 10 gl^{-1,} Tryptone: 10 gl⁻¹, and yeast extract: 5 gl⁻¹) in 15-ml tubes at 37° C. and 225 rpm. After 16 hours, the optical density (“OD”) of the culture is measured in a spectrophotometer (U-2100, Hitachi, Japan). The number of E. coli cells or the amount of DNA is calculated from an OD versus cell number or OD versus weight of DNA correlations. The culture is then diluted with distilled water to obtain varying numbers (10¹-10⁸) of E. coli cells per microlitre.

The E. coli cells are treated with a buffer (B1+B2, Magic Bead, USA) to lyse the cells and to release the DNA. Before the DNA is extracted, a pre-experimental wash is performed to wash and clean flow channel 34-3 by pumping distilled deionized water. After the DNA is extracted and the fluid is exhausted, a rinsing-before-elution operation is performed by pumping 10 mM Tris-HCI buffer (PH=7.6) through flow channel 34-3 of device 30 in order to remove any unbound substances. Then the beads are eluted by distilled deionized water to release the DNA into the solution. Referring to Table 1, Lanes 1 to 12 indicate the results of DNA extraction. Lanes 1 to 3 show the results of using no beads in a device in accordance with the present invention, for example, device 30, with the solution flowing back and forth. In Lane 1, serum is not added, while in Lanes 2 and 3, 25 μl and 50 μl of serum are respectively added. Since there are no DNA-extracting beads, there are no fluorescent bands in Lanes 1 to 3.

Lanes 4 to 8 show the results of using the device with free beads. In Lane 4, serum is not added, while in Lanes 5 and 6, 25 μl and 50 μl of serum are respectively added. Since there is no serum, the free beads can efficiently extract the DNA, as reflected by the corresponding fluorescent band. As to Lanes 5 and 6, when serum exists in the solution, however, the free beads can hardly extract any DNA because larger molecules, for example, protein, in the solution hinder the collision between the DNA and the beads. Lane 7 indicates the result of extracting the DNA from half, i.e., 5×10⁴ cells of the E. coli cells without serum in the solution. The fluorescence intensity is lower than that of Lane 4. The result of DNA extraction under static conditions without serum is indicated in Lane 8. The probability of collision between the DNA and the beads is decreased because there is no flow. Consequently, the fluorescence intensity of Lane 8 is lower than that of Lane 4.

Lane 9 shows the result of directly adding 0.5 ng, which corresponds to 10⁵ cells, of E. coli genomic DNA into a non-bead treated Eppendorf pipette, which is a known device for extracting DNA. The Eppendorf pipette with the fluid is spun in a vortex (VSM-3 Mixer, Shelton, USA) for 10 minutes. The fluorescence intensity of Lane 9 is the highest in FIG. 8A.

The experimental conditions for Lanes 10 to 12 are similar to those for Lanes 4 to 6, respectively, except that the beads are immobilized. Lanes 10 to 12 indicate the results of using device 30 with immobilized beads and the fluid flowing back and forth. The fluorescence intensity of Lane 10 is higher than that of Lane 4 (free beads) because the collision probability is higher. The fluorescence intensities of Lanes 11 and 12 are much higher than those of Lanes 5 and 6 (free beads), with similar amounts of serum existing in these lanes. It is apparent that the extraction efficiencies using immobilized beads are much higher than those using free beads.

FIG. 8B is a diagram showing a comparison of the experiment results shown in FIG. 8A. Referring to FIG. 8B, a quantitative analysis of the PCR product weight corresponding to each lane in FIG. 8A is illustrated. Lane 9 is the result of direct addition of E. coli genomic DNA into the Eppendorf. PCR product weight in Lane 9 is assigned to be 1.00 since it is the highest and corresponds to the genomic DNA weight of the same E. coli cell number, which is thus suitable to compare the efficiency of extraction in other lanes. Relative PCR product weights in the various lanes are defined as the percentage of the PCR product weight in Lane 9. With no serum present, the extraction efficiency of Lane 10 using the immobilized beads is approximately 2.1-fold higher than that of Lane 4 using free beads. Moreover, when serum is added, the average extraction efficiency of Lane 11 or 12 using immobilized beads is approximately 88-fold higher than that of Lane 5 or 6 using free beads, respectively. The results indicate that the extraction efficiency of the immobilized beads is higher than that of the free beads, especially when there is serum in the solution.

The foregoing disclosure of the preferred embodiments of the present invention has been presented for purposes of illustration and description. It is not intended to be exhaustive or to limit the invention to the precise forms disclosed. Many variations and modifications of the embodiments described herein will be apparent to one of ordinary skill in the art in light of the above disclosure. The scope of the invention is to be defined only by the claims appended hereto, and by their equivalents.

Further, in describing representative embodiments of the present invention, the specification may have presented the method and/or process of the present invention as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present invention should not be limited to the performance of their steps in the order written, and one skilled in the art can readily appreciate that the sequences may be varied and still remain within the spirit and scope of the present invention.

1. A device for extracting nucleic acids, comprising:
a chip; and
a flow channel of the chip including immobilized beads dispersed therein for extracting the nucleic acids.

2. The device of claim 1, further comprising a cover disposed over the chip.

3. The device of claim 2, wherein the cover includes an opening to communicate with a terminal of the chip for receiving a sample.

4. The device of claim 2, wherein the cover includes an opening to communicate with a terminal of the chip for receiving a buffer.

5. The device of claim 1, further comprising one of a pump or syringe for flowing a fluid in the flow channel bi-directionally.

6. The device of claim 1, wherein the flow channel includes projecting portions.

7. The device of claim 1, wherein the flow channel includes free beads dispersed therein, and the ratio of the surface area of the immobilized beads to that of the free beads is approximately 0.7.

8. The device of claim 2, further comprising a mixer disposed between the cover and the chip for generating a swirl.

9. The device of claim 8, wherein the cover includes a first opening and a second opening disposed diagonally with respect to the first opening.

10. The device of claim 8, wherein the mixer is removable from the chip.

11. A device for extracting nucleic acids, comprising:
a cover including a first opening and a second opening spaced apart from the first opening; and
a chip including a first terminal to communicate with the first opening and a second terminal to communicate with the second opening, the chip including a flow channel disposed between the first terminal and the second terminal,
wherein the flow channel includes an inner surface onto which immobilized beads are dispersed for binding with nucleic acids in a fluid flowing in the flow channel.

12. The device of claim 11, further comprising one of a pump or syringe for flowing the fluid in the flow channel backward and forward.

13. The device of claim 11, wherein the flow channel includes projecting portions.

14. The device of claim 1, wherein the flow channel includes free beads dispersed therein, and the ratio of the surface area of the immobilized beads to that of the free beads is approximately 0.7.

15. A device for extracting nucleic acids, comprising:
a chip including a flow channel;
a cover disposed over the chip including a first opening and a second opening diagonally disposed with respect to the first opening; and
a mixer disposed between the chip and the cover for mixing a sample and a buffer,
wherein the flow channel of the chip includes immobilized beads dispersed therein for extracting nucleic acids in a fluid containing the sample and the buffer.

16. The device of claim 15, further comprising an opening to communicate the mixer and the chip.

17. The device of claim 15, further comprising one of a pump or syringe for flowing the fluid in the flow channel bi-directionally.

18. The device of claim 15, wherein the flow channel includes projecting portions formed into the chip.

19. The device of claim 15, wherein the mixer generates a swirl in mixing the sample and the buffer.

20. The device of claim 15, wherein the mixer is removable from the substrate.

21. A method for extracting nucleic acids, comprising:
providing a chip;
providing the chip with a flow channel including an inner surface; and
dispersing immobilized beads for extracting the nucleic acids on the inner surface of the flow channel.

22. The method of claim 21, further comprising:
adding bead powder in deionized water;
injecting a solution of the bead powder and deionized water into the flow channel; and
shaking the solution in the flow channel.

23. The method of claim 21, further comprising flowing a fluid in the flow channel bi-directionally.

24. The method of claim 21, further comprising providing the flow channel with projecting portions formed into the chip.

25. The method of claim 21, further comprising mixing a sample and a buffer in a swirl before injecting the sample and buffer into the flow channel.

26. A method for extracting nucleic acids, comprising:
providing a flow channel;
providing a solution of bead powder and deionized water;
injecting the solution of bead powder and deionized water into the flow channel; and
shaking the solution in the flow channel to disperse immobilized beads in the flow channel.

27. The method of claim 26, further comprising flowing a fluid in the flow channel bi-directionally.

28. The method of claim 26, further comprising providing the flow channel with projecting portions.

29. The method of claim 26, further comprising generating a swirl of a fluid before injecting the fluid into the flow channel.

30. The method of claim 26, further comprising eluting the immobilized beads by deionized water to release the nucleic acids.
